(19) United States
(12) Patent Application Publication
(10) Pub. No.: US 2024/0324925 A1
(43) Pub. Date: Oct. 3, 2024

(54) APPARATUS AND METHOD FOR ANALYZING EFFICIENCY OF VIRTUAL TASK PERFORMANCE OF USER INTERACTING WITH EXTENDED REALITY

(71) Applicant: ELECTRONICS AND TELECOMMUNICATIONS RESEARCH INSTITUTE, Daejeon (KR)

(72) Inventors: Wook-Ho SON, Daejeon (KR); Jeung-Chul PARK, Daejeon (KR); Beom-Ryeol LEE, Daejeon (KR); Yong-Ho LEE, Daejeon (KR)

(73) Assignee: ELECTRONICS AND TELECOMMUNICATIONS RESEARCH INSTITUTE, Daejeon (KR)

(21) Appl. No.: 18/622,426

(22) Filed: Mar. 29, 2024

(30) Foreign Application Priority Data
Mar. 30, 2023 (KR) 10-2023-0041614

(51) Int. Cl. A61B 5/16; G02B 27/01; G06F 3/01

(52) U.S. Cl. CPC A61B 5/165; G02B 27/017; G06F 3/013; G06F 3/014; G06F 3/016; G06F 3/017; G02B 2027/0178

(57) ABSTRACT

Disclosed herein is an apparatus for analyzing efficiency of virtual task performance of a user interacting with eXtended Reality (XR). The apparatus includes memory in which at least one program is recorded and a processor for executing the program. The program may perform generating user interaction feature information from sensor information of a virtual reality (VR) device, calculating the quality of experience of a user as the values of multiple experience indices based on the feature information by applying a machine-learning model, and evaluating an experience based on a result of mapping the values of the multiple experience indices to generated metrics in order to analyze the effectiveness of the VR experience of the user.

### CROSS REFERENCE TO RELATED APPLICATION

This application claims the benefit of Korean Patent Application No. 10-2023-0041614, filed Mar. 30, 2023, which is hereby incorporated by reference in its entirety into this application.

### BACKGROUND OF THE INVENTION

### 1. Technical Field

The disclosed embodiment relates to technology for analyzing efficiency when a user wearing an eXtended Reality (XR) device performs a specific task based on various types of interactions in a virtual environment.

### 2. Description of the Related Art

Extended Reality (XR) is technology capable of freely selecting the use of Virtual Reality (VR) technology, Augmented Reality (AR) technology, or a combination thereof and creating extended reality using the selected technology. Extended reality is expected to be applied in various fields, such as education, healthcare, manufacturing, and the like.

However, there is no method capable of systematically validating the efficiency of the work performance of a user when the user wearing an XR device such as XR glasses performs a specific task or mission, such as education, training, or the like, based on user interactions in an XR environment.

For example, education based on existing online video conference platforms, such as Zoom, and the like, show poor educational effects due to limitations in communication, Zoom fatigue syndrome, and the like, but there is no method capable of systematizing the measurement of the effectiveness of such an education method and analyzing the result.

Also, discomfort of wearing an XR device or the low maturity of XR technology itself may cause various problems in the process of applying XR in education/training fields. Accordingly, in order to improve field applicability of XR technology depending on the problems that arise when XR technology is applied in the field, a method capable of systematically evaluating and analyzing the effectiveness for users is required.

In practice, existing VR/AR/XR/metaverse platforms do not provide any method capable of evaluating and improving user effectiveness. That is, although usefulness in the field provided by the state-of-the-art functions of hardware and software resources of such platforms is a major issue, there are no methods capable of validating the practicality.

### SUMMARY OF THE INVENTION

An object of the disclosed embodiment is to evaluate and analyze the Quality of Experience (QoE) of a user by quantifying the same when the user performs a task based on various interaction modalities in an XR environment.

Another object of the disclosed embodiment is to derive at least one treatment for improving the efficiency of virtual task performance of a user.

A further object of the disclosed embodiment is to propose a method capable of systematically analyzing and managing the effectiveness of work performance when a user performs work based on interactions with XR in various application fields, such as virtual education/training, and the like.

An apparatus for analyzing efficiency of virtual task performance of a user interacting with eXtended Reality (XR) according to an embodiment includes memory in which at least one program is recorded and a processor for executing the program. The program may perform generating user interaction feature information from sensor information of a virtual reality (VR) device, calculating the quality of experience of the user as values of multiple experience indices based on the feature information by applying a machine-learning model, and evaluating effectiveness of a VR experience of the user based on a result of mapping the values of the multiple experience indices to previously generated metrics.

Here, when generating the user interaction feature information, the program may construct a database by generating the interaction feature information based on spatial and time-series data.

Here, multiple interaction modalities may include a motion, eye gaze, and a sense of touch.

Here, the experience indices may include at least one of the degree of concentration, the degree of fatigue, the degree of interest, or the degree of arousal, or a combination thereof.

Here, the experience indices and the metrics may be generated based on domain knowledge of a given task.

Here, when evaluating the effectiveness, the program may generate the metrics based on the interrelationship between the experience indices and learning cognition attributes of the user.

Here, the program may further perform deriving at least one treatment based on a result of evaluation of the effectiveness of the VR experience.

Here, the VR device may include at least one of XR glasses, an eye-tracking device, or a haptic glove, or a combination thereof, provide virtual education and training simulation services based on virtual reality, and include a sensor for acquiring multimodal interaction information of at least one of a motion of the user, eye gaze of the user, or a sense of touch of the user, or a combination thereof.

A method for analyzing efficiency of virtual task performance of a user interacting with XR according to an embodiment may include generating user interaction feature information from sensor information of a VR device, calculating the quality of experience of the user as values of multiple experience indices based on the feature information by applying a machine-learning model, and evaluating effectiveness of a VR experience of the user based on a result of mapping the values of the multiple experience indices to previously generated metrics.

Here, generating the user interaction feature information may comprise constructing a database by generating the interaction feature information based on spatial and time-series data.

Here, multiple interaction modalities may include a motion, eye gaze, and a sense of touch.

Here, the experience indices may include at least one of the degree of concentration, the degree of fatigue, the degree of interest, or the degree of arousal, or a combination thereof.

Here, the experience indices and the metrics may be generated based on domain knowledge of a given task.

Here, evaluating the effectiveness may comprise generating the metrics based on the interrelationship between the experience indices and learning cognition attributes of the user.

Here, the method may further include deriving at least one treatment based on a result of evaluation of the effectiveness of the VR experience.

Here, the VR device may include at least one of XR glasses, an eye-tracking device, or a haptic glove, or a combination thereof, provide virtual education and training simulation services based on virtual reality, and include a sensor for acquiring multimodal interaction information of at least one of a motion of the user, eye gaze of the user, or a sense of touch of the user, or a combination thereof.

An apparatus for analyzing efficiency of virtual task performance of a user interacting with eXtended Reality (XR) according to an embodiment includes memory in which at least one program is recorded and a processor for executing the program. The program may perform generating user interaction feature information from sensor information of a VR device, constructing a feature information database by generating the interaction feature information based on spatial and time-series data, calculating the quality of experience of the user as values of multiple experience indices based on the feature information stored in the feature information database by applying a machine-learning model, generating metrics based on the interrelationship between the experience indices and learning cognition attributes of the user, mapping the values of the multiple experience indices to the metrics, evaluating experience based on the metrics, and deriving at least one treatment based on a result of evaluating effectiveness of virtual reality.

Here, multiple interaction modalities may include a motion, eye gaze, and a sense of touch.

Here, the experience indices may include at least one of the degree of concentration, the degree of fatigue, the degree of interest, or the degree of arousal, or a combination thereof.

Here, the experience indices and the metrics may be generated based on domain knowledge of a given task.

### BRIEF DESCRIPTION OF THE DRAWINGS

The above and other objects, features, and advantages of the present disclosure will be more clearly understood from the following detailed description taken in conjunction with the accompanying drawings, in which:

FIG. 1 is a schematic block diagram of an apparatus for analyzing efficiency of virtual task performance of a user interacting with eXtended Reality (XR) according to an embodiment;

FIG. 2 is an exemplary view of a QoE quantification unit according to an embodiment;

FIG. 3 is an exemplary view of metrics for experience indices according to an embodiment;

FIG. 4 is an exemplary view of an experience effectiveness evaluation unit according to an embodiment;

FIG. 5 is a flowchart for explaining a method for analyzing efficiency of virtual task performance of a user interacting with eXtended Reality (XR) according to an embodiment;

FIG. 6 is a flowchart for explaining a step for generating user interaction feature information according to an embodiment;

FIG. 7 is a flowchart for explaining a step for evaluating an experience according to an embodiment; and

FIG. 8 is a view illustrating a computer system configuration according to an embodiment.

### DESCRIPTION OF THE PREFERRED EMBODIMENTS

The advantages and features of the present disclosure and methods of achieving them will be apparent from the following exemplary embodiments to be described in more detail with reference to the accompanying drawings. However, it should be noted that the present disclosure is not limited to the following exemplary embodiments, and may be implemented in various forms. Accordingly, the exemplary embodiments are provided only to disclose the present disclosure and to let those skilled in the art know the category of the present disclosure, and the present disclosure is to be defined based only on the claims. The same reference numerals or the same reference designators denote the same elements throughout the specification.

It will be understood that, although the terms “first,” “second,” etc. may be used herein to describe various elements, these elements are not intended to be limited by these terms. These terms are only used to distinguish one element from another element. For example, a first element discussed below could be referred to as a second element without departing from the technical spirit of the present disclosure.

The terms used herein are for the purpose of describing particular embodiments only and are not intended to limit the present disclosure. As used herein, the singular forms are intended to include the plural forms as well, unless the context clearly indicates otherwise. It will be further understood that the terms “comprises,” “comprising,”, “includes” and/or “including,” when used herein, specify the presence of stated features, integers, steps, operations, elements, and/or components, but do not preclude the presence or addition of one or more other features, integers, steps, operations, elements, components, and/or groups thereof.

Unless differently defined, all terms used herein, including technical or scientific terms, have the same meanings as terms generally understood by those skilled in the art to which the present disclosure pertains. Terms identical to those defined in generally used dictionaries should be interpreted as having meanings identical to contextual meanings of the related art, and are not to be interpreted as having ideal or excessively formal meanings unless they are definitively defined in the present specification.

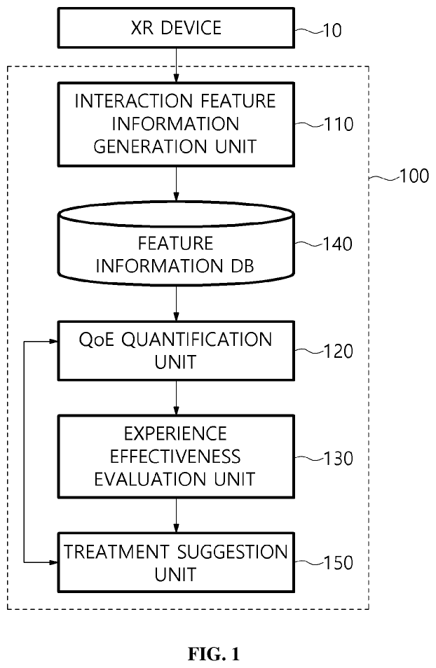

Hereinafter, an apparatus and method for analyzing efficiency of virtual task performance of a user interacting with extended reality according to an embodiment will be described in detail with reference to FIGS. 1 to 8.

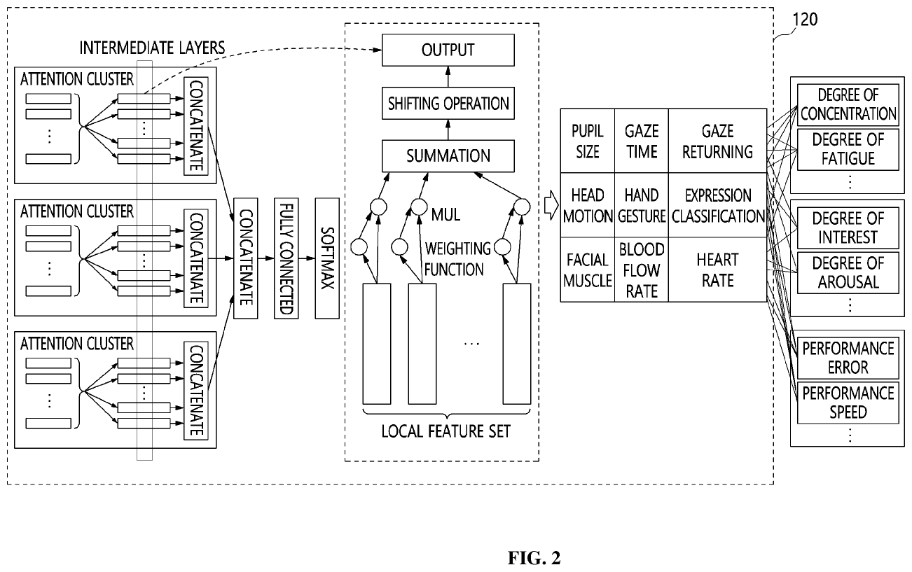

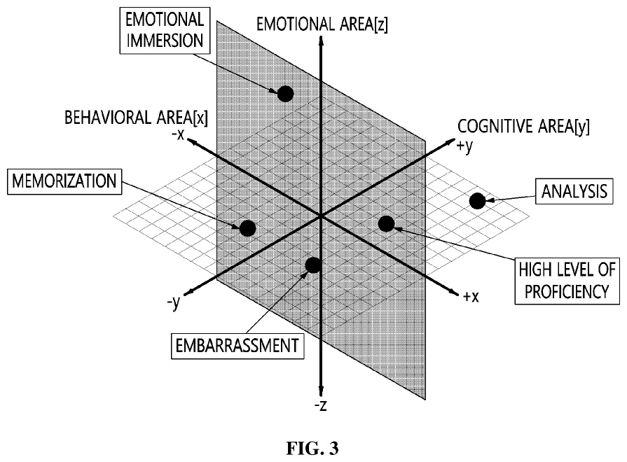

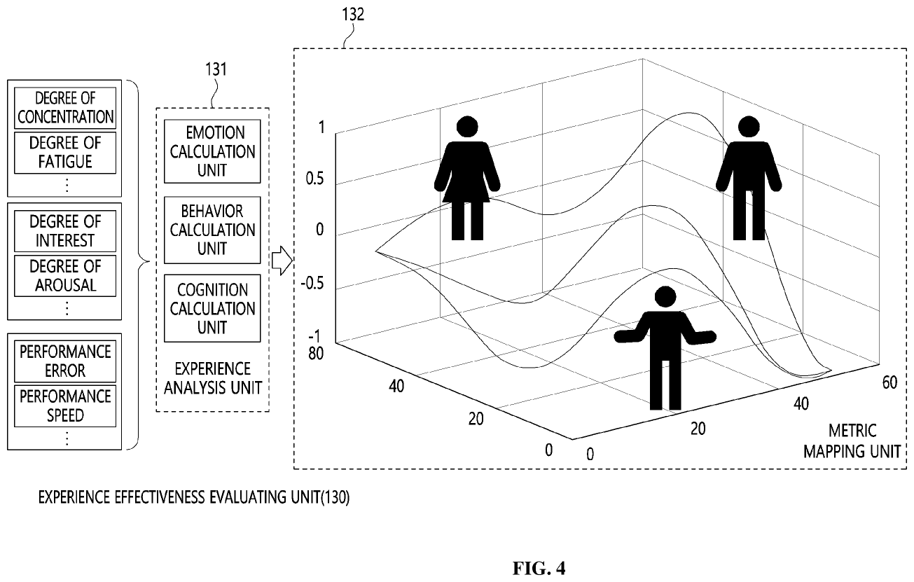

FIG. 1 is a schematic block diagram of an apparatus for analyzing efficiency of virtual task performance of a user interacting with eXtended Reality (XR) according to an embodiment, FIG. 2 is an exemplary view of a QoE quantification unit according to an embodiment, FIG. 3 is an exemplary view of metrics for experience indices according to an embodiment, and FIG. 4 is an exemplary view of an experience effectiveness evaluation unit according to an embodiment.

Referring to FIG. 1, the apparatus 100 for analyzing efficiency of virtual task performance of a user interacting with extended reality according to an embodiment may evaluate the effectiveness of a virtual experience and suggest at least one treatment when a user wearing an XR device 10 performs a task based on interactions with extended reality.

Here, the task based on interactions with extended reality may be virtual education/training simulations based on virtual reality for realistic education of subjects, such as science, mathematics, English, and the like, and for various types of virtual training in a medical field, a military field, a manufacturing field, and the like.

Here, the user may be a student or trainee performing the virtual education or training simulation.

The XR device 10 may include XR glasses, an eye-tracking device, a haptic glove, and the like worn by the user. The XR device 10 includes various kinds of sensors attached thereto, thereby acquiring information about various modality interactions, which is sensing information, such as the motion, the eye gaze, the sense of touch, and the like of the user.

Specifically, the apparatus 100 for analyzing efficiency of virtual task performance of a user interacting with extended reality according to an embodiment (referred to as the ‘apparatus’ hereinbelow) may include an interaction feature information generation unit 110, a QoE quantification unit 120, and an experience effectiveness evaluation unit 130. Also, the apparatus 100 may further include a feature information database (DB) 140 and a treatment suggestion unit 150.

The interaction feature information generation unit 110 extracts the sensing information of the XR device 10 and derives interaction feature information of a user therefrom. That is, using the information about various modality interactions, including a motion, eye gaze, a sense of touch, and the like, feature information may be generated so as to match the characteristics of each of the interactions. This may be the process of preprocessing the data to be processed by the machine-learning model of the QoE quantification unit 120.

The interaction feature information derived as described above may be stored in the feature information DB 140.

The QoE quantification unit 120 may quantify the Quality of Experience (QoE) of a user interacting with extended reality based on the machine-learning model that receives the generated interaction feature information as input.

The QoE quantification unit 120 based on the machine-learning model outputs estimations by estimating the QoE of the user in the form of values of various experience indices.

Here, the various experience indices may include the degree of concentration, the degree of fatigue, the degree of interest, the degree of arousal, a performance error, a performance speed, and the like, as illustrated in FIG. 2. These may be indices for representing the kinds of user experiences that a user can express when the user experiences the XR environment based on various interactions.

Here, the experience indices may be set based on the knowledge of the domain of a task based on interactions with extended reality. Here, the domain may include language, science, medical care, military/security training, and the like. For example, the degree of interest, the degree of arousal, the degree of concentration, the degree of fatigue, and the like may be set as the experience indices in the science domain, and the degree of concentration, the degree of fatigue, the performance error, the performance speed, and the like may be set as the experience indices in the case of military training.

Also, the machine-learning model may use a machine-learning model based on an attention mechanism, as illustrated in FIG. 2.

The attention mechanism is a method for selecting values to which more focus is to be given in an encoder when output is predicted. Because existing seq2seq depends only on the final result of an encoder, it can be seen that output at every time point is predicted based on the same information. Therefore, it can be seen that seq2seq does not adequately reflect the current state. In order to prevent this problem, the attention mechanism is configured such that the output of an encoder at every time point is compared with the current state and then a weight is given to the most similar value.

In order to estimate the respective values of the experience indices, the machine-learning model based on the attention mechanism may be formed using an algorithm in which weights for factors including the pupil size, the gaze time, the gaze returning, the head motion, the hand gesture, the expression classification, the facial muscle, the blood flow rate, the heart rate, and the like of a user are extracted from interaction feature information and the respective values of the experience indices are estimated from the weights.

The weights for the factors, including the pupil size, the gaze time, the gaze returning, the head motion, the hand gesture, the expression classification, the facial muscle, the blood flow rate, the heart rate, and the like, calculated in the intermediate process of the machine-learning model based on the attention mechanism may be used to derive at least one treatment by being traced back by the treatment suggestion unit 150. However, this is an example of the present disclosure, and the present disclosure is not limited thereto. That is, the machine-learning model according to an embodiment may use any of various algorithms other than the attention mechanism.

The experience effectiveness evaluation unit 130 may generate metrics for analyzing the effectiveness of an XR experience of the user and evaluate the effectiveness of the experience based on the result of mapping the values of the XR experience indices to the generated metrics.

Here, the metrics for analyzing the effectiveness of the XR experience may be generated based on multiple perception attributes. For example, the metrics for analyzing the effectiveness of the XR experience may be generated in a three-dimensional (3D) coordinate space formed of an x-axis, a y-axis, and a z-axis, as illustrated in FIG. 3.

Here, the x-axis, the y-axis, and the z-axis may correspond to multiple perception attributes, e.g., a behavioral attribute, a cognitive attribute, and an emotional attribute, as illustrated in FIG. 3.

Here, the multiple perception attributes may be set based on the knowledge of education/training domains, which are fields in which XR interactions are applied.

Referring to FIG. 4, the experience effectiveness evaluation unit 130 may include an experience analysis unit 131 and a metric mapping unit 132.

The experience analysis unit 131 may calculate the respective values of the multiple perception attributes forming the metrics based on the values of the XR experience indices, including the degree of concentration, the degree of fatigue, the degree of interest, the degree of arousal, and the like of the user output from the QoE quantification unit 120. These may be calculated based on the interrelationship between the XR experience indices and the perception attributes.

For example, the experience analysis unit 131 may include an emotion calculation unit, a behavior calculation unit, and a cognition calculation unit. The emotion calculation unit, the behavior calculation unit, and the cognition calculation unit may calculate the respective values for the emotional attribute, the behavioral attribute, and the cognitive attribute using a predetermined equation by receiving the values of the XR experience indices. The predetermined equation may be set differently depending on the domain of the task based on XR interactions.

The metric mapping unit 132 maps the calculated emotional attribute value, the calculated behavioral attribute value, and the calculated cognitive attribute value to the metrics. That is, the calculated values are mapped to 3D coordinates corresponding to (the behavioral attribute value, the cognitive attribute value, the emotional attribute value) in the 3D coordinate space, such as that illustrated in FIG. 3.

The experience effectiveness evaluation unit 130 evaluates the efficiency of virtual task performance of the user interacting with extended reality based on the result of mapping to the metrics. For example, referring to FIG. 3, the result of virtual task performance of the user may be evaluated as emotional immersion, memorization, embarrassment, a high level of proficiency, analysis, or the like depending on the locations of the metrics.

Referring again to FIG. 1, the treatment suggestion unit 150 may suggest at least one treatment for improving effectiveness by analyzing the factors depending on the result of evaluation of the experience effectiveness evaluation unit 130.

Here, the treatment suggestion unit 150 may suggest at least one treatment by tracing back the weights for the factors, including the pupil size, the gaze time, the gaze returning, the head motion, the hand gesture, the expression classification, the facial muscle, the blood flow rate, the heart rate, and the like, calculated in the intermediate process of the attention-mechanism-based machine-learning model of the experience effectiveness evaluation unit 130, as described above.

For example, when virtual task performance of the user is evaluated as a lack of concentration and when it is analyzed that it is necessary to reduce a head motion as the result of tracing back the attention-mechanism-based machine-learning model of the experience effectiveness evaluation unit 130, a treatment to adjust the audience density may be suggested. When virtual task performance of the user is evaluated as a lack of emotional immersion and when it is analyzed that it is necessary to increase hand gestures as the result of tracing back the attention-mechanism-based machine-learning model of the experience effectiveness evaluation unit 130, a treatment to adjust object settings may be suggested. When virtual task performance of the user is evaluated as a high level of proficiency and when it is analyzed that it is necessary to increase gaze time as the result of tracing back the attention-mechanism-based machine-learning model of the experience effectiveness evaluation unit 130, a treatment to adjust the degree of difficulty of a task may be suggested. When virtual task performance of the user is evaluated as embarrassment and when it is analyzed that it is necessary to reduce gaze distraction as the result of tracing back the attention-mechanism-based machine-learning model of the experience effectiveness evaluation unit 130, a treatment to adjust an interface may be suggested. However, the treatments described above are merely examples for helping understanding, and the present disclosure is not limited thereto.

The treatments may be output using a display means or a speaker such that the user recognizes the treatments.

Meanwhile, the apparatus 100 may suggest at least one treatment by evaluating effectiveness in real time while the virtual task is being performed. Alternatively, the apparatus 100 may suggest the at least one treatment by evaluating effectiveness at predetermined intervals while the virtual task is being performed.

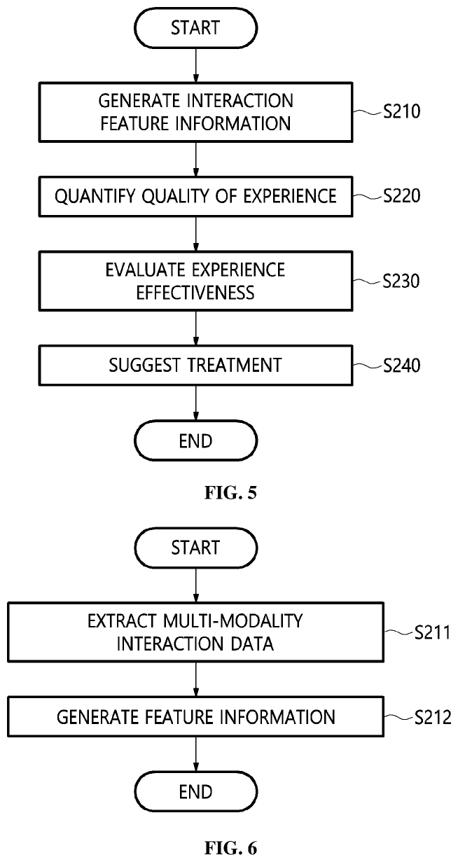

FIG. 5 is a flowchart for explaining a method for analyzing efficiency of virtual task performance of a user interacting with extended reality according to an embodiment.

Referring to FIG. 5, the method for analyzing efficiency of virtual task performance of a user interacting with extended reality according to an embodiment may include generating user interaction feature information from sensor information of a VR device at step S210, calculating the Quality of Experience (QoE) of a user as values of multiple experience indices based on the feature information by applying a machine-learning model at step S220, and evaluating an experience based on a result of mapping the values of the multiple experience indices to generated metrics in order to analyze the effectiveness of the VR experience of the user at step S230.

The method for analyzing efficiency of virtual task performance of a user interacting with extended reality according to an embodiment may further include deriving at least one treatment based on the result of evaluation of the effectiveness of virtual reality at step S240.

FIG. 6 is a flowchart for explaining the step (S210) of generating user interaction feature information according to an embodiment.

Referring to FIG. 6, the apparatus 100 extracts multi-modality interaction data, including a motion, eye gaze, a sense of touch, expression, bio signal and the like, from the XR device 10 at step S211. Here, the apparatus 100 extracts raw time-series data as multi-modality interaction data modalities at step S211.

The apparatus 100 derives interaction feature information of the user from the extracted raw time-series data at step S212. Here, the feature information may be generated so as to match the characteristics of each interaction. This may be the process of preprocessing the data to be processed by the machine-learning model at the step (S220) of quantify the QoE.

Here, the derived interaction feature information may be stored in the feature information DB 140.

Meanwhile, at the step (S220) of calculating the QoE of the user as the values of multiple experience indices based on the feature information by applying the machine-learning model according to an embodiment, the QoE of the user for the interaction with extended reality may be quantified based on the machine-learning model that receives the generated interaction feature information as input. That is, the QoE of the user may be predicted in the form of the values of various experience indices.

Here, the various experience indices may include the degree of concentration, the degree of fatigue, the degree of interest, the degree of arousal, a performance error, a performance speed, and the like. These may be indices for representing the kinds of user experiences that a user can express when the user experiences the XR environment based on various interactions.

Here, the experience indices may be set based on the knowledge of the domain of a task based on interactions with extended reality. Here, the domain may include language, science, medical care, military/security training, and the like. For example, the degree of interest, the degree of arousal, the degree of concentration, the degree of fatigue, and the like may be set as experience indices in the science domain, and the degree of concentration, the degree of fatigue, the performance error, the performance speed, and the like may be set as experience indices in the case of military training.

Here, the machine-learning model may use a machine-learning model based on an attention mechanism.

In order to estimate the respective values of the experience indices, the machine-learning model based on the attention mechanism may be formed using an algorithm in which weights for factors including the pupil size, the gaze time, the gaze returning, the head motion, the hand gesture, the expression classification, the facial muscle, the blood flow rate, the heart rate, and the like of a user are extracted from interaction feature information and the respective values of the experience indices are estimated from the weights.

The weights for the factors, including the pupil size, the gaze time, the gaze returning, the head motion, the hand gesture, the expression classification, the facial muscle, the blood flow rate, the heart rate, and the like, calculated in the intermediate process of the machine-learning model based on the attention mechanism may be used to derive at least one treatment by being traced back by the treatment suggestion unit 150. However, this is an example of the present disclosure, and the present disclosure is not limited thereto. That is, the machine-learning model according to an embodiment may use any of various algorithms other than the attention mechanism.

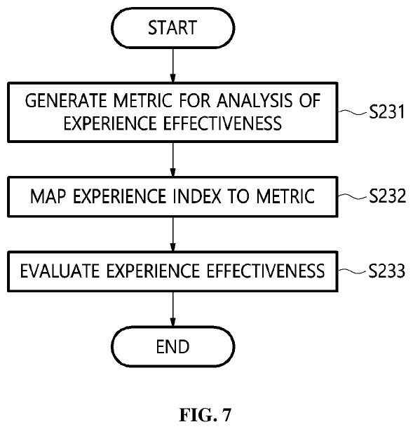

FIG. 7 is a flowchart for explaining the step (S230) of evaluating effectiveness of an experience according to an embodiment.

Referring to FIG. 7, the apparatus 100 may generate metrics for analyzing the effectiveness of the XR experience of the user at step S231 and evaluate the effectiveness of the experience at step S233 based on a result of mapping the values of the XR experience indices to the generated metrics, which is performed at step S232.

Here, the metrics for analyzing the effectiveness of the XR experience may be generated based on multiple perception attributes. For example, the metrics for analyzing the effectiveness of the XR experience may be generated in a 3D coordinate space formed of an x-axis, a y-axis, and a z-axis.

Here, the x-axis, the y-axis, and the z-axis may correspond to multiple perception attributes, e.g., a behavioral attribute, an emotional attribute, and a cognitive attribute.

Here, the multiple perception attributes may be set based on the knowledge of education/training domains, which are fields in which XR interactions are applied.

When it maps the values of the XR experience indices to the generated metrics at step S232, the apparatus 100 may calculate the respective values of the multiple perception attributes forming the metrics based on the values of the XR experience indices, including the degree of concentration, the degree of fatigue, the degree of interest, the degree of arousal, and the like of the user. These may be calculated based on the interrelationship between the XR experience indices and the perception attributes. Here, the respective values for the cognitive attribute, the behavioral attribute, and the emotional attribute may be calculated using a predetermined equation by receiving the values of the experience indices as input. The predetermined equation may be set differently depending on the domain of the task based on XR interactions.

Subsequently, the apparatus 100 maps the calculated emotional attribute value, the calculated behavioral attribute value, and the calculated cognitive attribute value to the metrics. That is, the calculated values are mapped to the 3D coordinate point, that is, (the behavioral attribute value, the cognitive attribute value, the emotional attribute value), in the 3D coordinate space.

Subsequently, when it evaluates the effectiveness of the experience at step S233, the apparatus 100 evaluates the efficiency of virtual task performance of the user interacting with extended reality based on the result of mapping to the metrics. For example, the result of virtual task performance of the user may be evaluated as emotional immersion, memorization, embarrassment, a high level of proficiency, analysis, or the like depending on the locations of the metrics.

Referring again to FIG. 5, the apparatus 100 may suggest at least one treatment for improving effectiveness by tracking the factors causing the evaluation result at the step (S240) of deriving at least one treatment based on the result of evaluation of the effectiveness of virtual reality.

Here, the at least one treatment may be suggested by tracing back the weights for the factors, including the pupil size, the gaze time, the gaze returning, the head motion, the hand gesture, the expression classification, the facial muscle, the blood flow rate, the heart rate, and the like, calculated in the intermediate process of the machine-learning model based on the attention mechanism at the step (S230) of evaluating the effectiveness of the experience, as described above.

For example, when virtual task performance of the user is evaluated as a lack of concentration and when it is analyzed that it is necessary to reduce a head motion as the result of tracing back the machine-learning model based on the attention mechanism at the step (S230) of evaluating the effectiveness of the experience, a treatment to adjust the audience density may be suggested. When virtual task performance of the user is evaluated as a lack of emotional immersion and when it is analyzed that it is necessary to increase hand gestures as the result of tracing back the machine-learning model based on the attention mechanism at the step (S230) of evaluating the effectiveness of the experience, a treatment to adjust object settings may be suggested. When virtual task performance of the user is evaluated as a high level of proficiency and when it is analyzed that it is necessary to increase gaze time as the result of tracing back the machine-learning model based on the attention mechanism at the step (S230) of evaluating the effectiveness of the experience, a treatment to adjust the degree of difficulty of a task may be suggested. When virtual task performance of the user is evaluated as embarrassment and when it is analyzed that it is necessary to reduce gaze distraction as the result of tracing back the machine-learning model based on the attention mechanism at the step (S230) of evaluating the effectiveness of the experience, a treatment to adjust an interface may be suggested. However, the treatments described above are merely examples for helping understanding, and the present disclosure is not limited thereto.

The treatments may be output using a display means or a speaker such that the user recognizes the treatments.

Meanwhile, the apparatus 100 may suggest at least one treatment by evaluating effectiveness in real time while the virtual task is being performed. Alternatively, the apparatus 100 may suggest the at least one treatment by evaluating effectiveness at predetermined intervals while the virtual task is being performed.

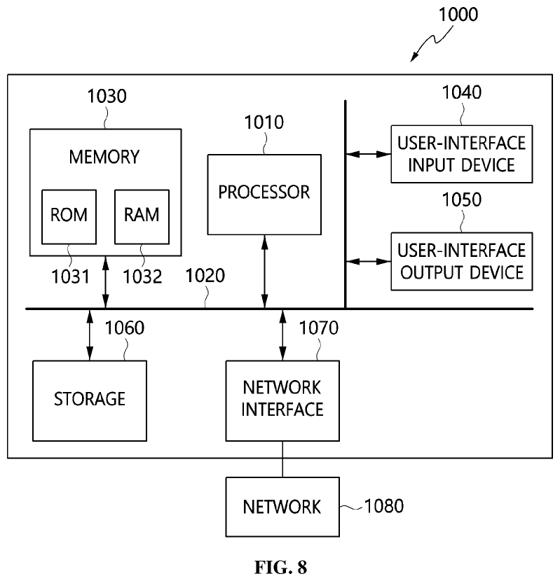

FIG. 8 is a view illustrating a computer system configuration according to an embodiment.

The apparatus 100 for analyzing efficiency of virtual task performance of a user interacting with extended reality according to an embodiment may be implemented in a computer system 1000 including a computer-readable recording medium.

The computer system 1000 may include one or more processors 1010, memory 1030, a user-interface input device 1040, a user-interface output device 1050, and storage 1060, which communicate with each other via a bus 1020. Also, the computer system 1000 may further include a network interface 1070 connected to a network 1080. The processor 1010 may be a central processing unit or a semiconductor device for executing a program or processing instructions stored in the memory 1030 or the storage 1060. The memory 1030 and the storage 1060 may be storage media including at least one of a volatile medium, a nonvolatile medium, a detachable medium, a non-detachable medium, a communication medium, or an information delivery medium, or a combination thereof. For example, the memory 1030 may include ROM 1031 or RAM 1032.

The disclosed embodiment may evaluate and analyze the Quality of Experience (QoE) of a user by quantifying the same when the user performs a task based on various interaction modalities in an XR environment.

The disclosed embodiment may derive at least one treatment for improving the efficiency of virtual task performance of a user.

The disclosed embodiment may propose a method capable of systematically analyzing and managing the effectiveness of work performance when a user performs work based on interactions with extended reality in various application fields, such as virtual education/training, and the like.

Although embodiments of the present disclosure have been described with reference to the accompanying drawings, those skilled in the art will appreciate that the present disclosure may be practiced in other specific forms without changing the technical spirit or essential features of the present disclosure. Therefore, the embodiments described above are illustrative in all aspects and should not be understood as limiting the present disclosure.

What is claimed is:

1. An apparatus for analyzing efficiency of virtual task performance of a user interacting with eXtended Reality (XR), comprising:
memory in which at least one program is recorded; and
a processor for executing the program,
wherein the program performs
generating user interaction feature information from sensor information of a virtual reality (VR) device,
calculating quality of experience of the user as values of multiple experience indices based on the feature information by applying a machine-learning model, and
evaluating effectiveness of a VR experience of the user based on a result of mapping the values of the multiple experience indices to previously generated metrics.

2. The apparatus of claim 1, wherein, when generating the user interaction feature information, the program constructs a database by generating the interaction feature information based on spatial and time-series data.

3. The apparatus of claim 2, wherein multiple interaction modalities include a motion, eye gaze, and a sense of touch.

4. The apparatus of claim 1, wherein the experience indices include at least one of a degree of concentration, a degree of fatigue, a degree of interest, or a degree of arousal, or a combination thereof.

5. The apparatus of claim 1, wherein the experience indices and the metrics are generated based on domain knowledge of a given task.

6. The apparatus of claim 1, wherein, when evaluating the effectiveness, the program generates the metrics based on an interrelationship between the experience indices and learning cognition attributes of the user.

7. The apparatus of claim 1, wherein the program further performs deriving at least one treatment based on a result of evaluation of the effectiveness of the VR experience.

8. The apparatus of claim 1, wherein the VR device includes at least one of XR glasses, an eye-tracking device, or a haptic glove, or a combination thereof, provides virtual education and training simulation services based on virtual reality, and includes a sensor for acquiring multimodal interaction information of at least one of a motion of the user, eye gaze of the user, or a sense of touch of the user, or a combination thereof.

9. A method for analyzing efficiency of virtual task performance of a user interacting with eXtended Reality (XR), comprising:
generating user interaction feature information from sensor information of a virtual reality (VR) device;
calculating quality of experience of the user as values of multiple experience indices based on the feature information by applying a machine-learning model; and
evaluating effectiveness of a VR experience of the user based on a result of mapping the values of the multiple experience indices to previously generated metrics.

10. The method of claim 9, wherein generating the user interaction feature information comprises constructing a database by generating the interaction feature information based on spatial and time-series data.

11. The method of claim 10, wherein multiple interaction modalities include a motion, eye gaze, and a sense of touch.

12. The method of claim 11, wherein the experience indices include at least one of a degree of concentration, a degree of fatigue, a degree of interest, or a degree of arousal, or a combination thereof.

13. The method of claim 9, wherein the experience indices and the metrics are generated based on domain knowledge of a given task.

14. The method of claim 9, wherein evaluating the effectiveness comprises generating the metrics based on an interrelationship between the experience indices and learning cognition attributes of the user.

15. The method of claim 9, further comprising:
deriving at least one treatment based on a result of evaluation of the effectiveness of the VR experience.

16. The method of claim 9, wherein the VR device includes at least one of XR glasses, an eye-tracking device, or a haptic glove, or a combination thereof, provides virtual education and training simulation services based on virtual reality, and includes a sensor for acquiring multimodal interaction information of at least one of a motion of the user, eye gaze of the user, or a sense of touch of the user, or a combination thereof.

17. An apparatus for analyzing efficiency of virtual task performance of a user interacting with eXtended Reality (XR), comprising:
memory in which at least one program is recorded; and
a processor for executing the program,
wherein the program performs
generating user interaction feature information from sensor information of a virtual reality (VR) device,
constructing a feature information database by generating the interaction feature information based on spatial and time-series data,
calculating quality of experience of the user as values of multiple experience indices based on the feature information stored in the feature information database by applying a machine-learning model,
generating metrics based on an interrelationship between the experience indices and learning cognition attributes of the user,
mapping the values of the multiple experience indices to the metrics,
evaluating an experience based on the metrics, and
deriving at least one treatment based on a result of evaluating effectiveness of virtual reality.

18. The apparatus of claim 17, wherein multiple interaction modalities include a motion, eye gaze, and a sense of touch.

19. The apparatus of claim 17, wherein the experience indices include at least one of a degree of concentration, a degree of fatigue, a degree of interest, or a degree of arousal, or a combination thereof.

20. The apparatus of claim 19, wherein the experience indices and the metrics are generated based on domain knowledge of a given task.
